(19) United States
(12) Patent Application Publication
(10) Pub. No.: US 2026/0243166 A1
(43) Pub. Date: Aug. 20, 2026

(54) TRIAXIAL STRESS SIMULATION METHOD FOR WATER STORAGE ROADWAYS IN ABANDONED MINE PUMPED STORAGE

(71) Applicants: GUIZHOU UNIVERSITY, Guiyang (CN); SHANDONG UNIVERSITY OF SCIENCE AND TECHNOLOGY, Qingdao (CN)

(72) Inventors: Zhijie WEN, Guiyang (CN); Jiazheng ZHANG, Guiyang (CN); Zhongfu CHEN, Guiyang (CN); Zhenqi SONG, Guiyang (CN); Zhengmeng HOU, Guiyang (CN); Guangming ZHAO, Guiyang (CN); Pengfei JIANG, Guiyang (CN); Qingsong LI, Guiyang (CN)

(21) Appl. No.: 19/540,514

(22) Filed: Feb. 13, 2026

(30) Foreign Application Priority Data
Feb. 19, 2025 (CN) 202510184760.0

(51) Int. Cl. E21B 49/08; E21B 49/00; E21B 49/02; G01N 3/30; G01N 33/24

(52) U.S. Cl. CPC E21B 49/0875; E21B 49/00; E21B 49/006; E21B 49/02; G01N 3/30; G01N 33/24; G01N 2203/0019; G01N 2203/0048; G01N 2203/0256

(57) ABSTRACT

Disclosed is a triaxial stress simulation method for water storage roadways in abandoned mine pumped storage. The method relates technical field of stress simulation for water storage roadways in abandoned mine pumped storage. The method includes: preparing rock samples and conducting uniaxial compression tests to obtain the maximum failure load; installing another rock sample into a true triaxial dynamic loading system and applying preload for fixing; applying maximum principal stress, intermediate principal stress, and minimum principal stress to the rock sample separately; keeping the three-dimensional principal stress constant, applying single-sided dynamic water pressure to the rock sample, and applying dynamic load to the rock sample through an impact transmission rod; recording and analyzing stress-strain curve data, stress-time curve data, and water pressure-time curve data. The real state of the pumped storage and water storage roadways in an abandoned mine can be simulated more truly.

### CROSS-REFERENCE TO RELATED APPLICATIONS

This application claims priority to Chinese Application No. 202510184760.0, filed on Feb. 19, 2025, entitled “TRIAXIAL STRESS SIMULATION METHOD FOR WATER STORAGE ROADWAYS IN ABANDONED MINE PUMPED STORAGE”. These contents are hereby incorporated by reference.

### TECHNICAL FIELD

The present disclosure relates to the technical field of stress simulation for water storage roadways in abandoned mine pumped storage, and in particular to a triaxial stress simulation method for water storage roadways in abandoned mine pumped storage.

### BACKGROUND

With the transformation of energy structure and the demand for sustainable development, pumped storage, as an important energy storage technology, is playing an increasingly important role in the energy system. Abandoned mines have become potential application sites for pumped storage projects due to their abundant underground space and stable geological conditions. By transforming abandoned mines into pumped storage power stations, not only the idle resources can be effectively utilized, but traditional energy supply pressure can also be alleviated, and the stability and scheduling flexibility of the power system can be improved. Therefore, the abandoned mine pumped storage project has broad application prospects in the future energy industry.

In the design and construction of water storage roadways in abandoned mine pumped storage, the triaxial stress state borne by the water storage roadways plays a crucial role in the structural stability and water tightness. The triaxial stress state of the water storage roadways is mainly influenced by the combined effects of the geostress field of the surrounding rock mass, water pressure, and the excavation stress of the mine. Therefore, accurately simulating the triaxial stress state inside the roadway, especially the interaction between water pressure and rock mass, is crucial for evaluating the stability and design rationality of the roadway. Although various rock mechanics testing methods have been adopted in the existing technology, due to the inability to fully simulate the complex water-rock-force interactions in water storage roadways of the abandoned mine, it is often difficult to accurately predict the mechanical behavior and failure mode of rocks during the water storage process.

Thus, the existing technology needs to be further improved.

### SUMMARY

The present disclosure aims to provide a triaxial stress simulation method for water storage roadways in abandoned mine pumped storage, which can reproduce the complex triaxial stress state inside the roadway in a laboratory environment and more realistically simulate the real state of water storage roadways in abandoned mine pumped storage.

In order to achieve the above objectives, the present disclosure adopts the following technical solutions:

The triaxial stress simulation method for water storage roadways in abandoned mine pumped storage, including the following steps in sequence:
a, Preparing a cube sample as a rock sample, and carrying out an uniaxial compression test on the rock sample to obtain a maximum failure load of the rock sample;
b, Installing another rock sample in a true triaxial dynamic loading system, wherein the true triaxial dynamic loading system includes a workbench, a main frame, a confining chamber, a vertical loading unit, a horizontal loading unit, a longitudinal loading unit, a lifting device, and a feeding and discharging platform; the vertical loading unit includes an impact transmission rod; the longitudinal loading unit includes a front loading oil cylinder, a rear loading oil cylinder, a front loading head, and a rear loading head; the front loading head and the rear loading head are arranged opposite each other from front to rear inside the confining chamber through a longitudinal loading rod, the front loading oil cylinder and the rear loading oil cylinder are symmetrically arranged on the front side and rear side of the main frame through a rotating arm, and static loads are synchronously applied to the front loading head and the rear loading head;

A square groove is formed on an end face of the front loading head, and a water channel is arranged inside the longitudinal loading rod connected to the front loading head, and the water channel is connected to a water supply device for providing high-pressure water into the square groove;
c. Applying a preload to fix the rock sample in the true triaxial dynamic loading system; then applying a maximum principal stress σ1, an intermediate principal stress σ2, and a minimum principal stress σ3 to the rock sample through the vertical loading unit, the horizontal loading unit, and longitudinal loading unit, respectively; during an initial loading process, the maximum principal stress σ1 and the intermediate principal stress σ2 are 50% of the maximum failure load specified in step a; the minimum principal stress σ3 is 10% of the maximum failure load specified in step a; after the loading stabilizes, the maximum principal stress σ1 is adjusted to 80% of the maximum failure load specified in step a.
d, Keeping the maximum principal stress σ1, the intermediate principal stress σ2 and the minimum principal stress σ3 unchanged, applying single-sided dynamic water pressure to the rock sample through the front loading head, and simultaneously applying dynamic load to the rock sample through the impact transmission rod;
e. Observing and recording; ending the test when the rock sample in the true triaxial dynamic loading system has failed, recording obtained stress-strain curve data, stress-time curve data, and water pressure-time curve data, and observing a failure mode of the rock sample;
f, Combined with theoretical knowledge, obtaining the failure strength and failure form of the surrounding rock of water storage roadways in abandoned mine pumped storage under different depths and water pressures.

In step a of the above simulation method, a specification of the rock sample is 150 mm×150 mm×150 mm.

The triaxial stress simulation method for water storage roadways in abandoned mine pumped storage mentioned above, in step b, the vertical loading unit further includes an impact loading hydraulic cylinder, top loading hydraulic cylinders, and a top loading head; the impact loading hydraulic cylinder is vertically and fixedly installed at a center of a top of the main frame, a plurality of the top loading hydraulic cylinders are provided, all of the top loading hydraulic cylinders are regularly arranged around the impact loading hydraulic cylinder, and execution ends of the top loading hydraulic cylinders are fixedly connected by a force collecting block;

The top loading head is arranged on an upper part of an inner side of the confining chamber through a vertical loading rod, the impact transmission rod is coaxially arranged inside the vertical loading rod, the force collecting block applies static load to the top loading head through the vertical loading rod, and the impact loading hydraulic cylinder applies impact load to the top loading head through the impact transmission rod, wherein the impact load applied is true triaxial stress.

In step c of the above simulation method, the preload in the Z-axis direction is applied to the rock sample through the vertical loading unit, and the preload is 10 kN; then the preload in X-axis direction and Y-axis direction are applied to the rock sample by the longitudinal loading unit and the horizontal loading unit respectively, and the preload is 10 kN.

In step d of the above simulation method, the dynamic load applied to the rock sample by the impact transmission rod is 5% of the maximum failure load in step a.

In step e of the above simulation method, the rock sample is determined whether it is damaged by stress-strain curve and the water pressure-time curve.

In the above simulation method, when the maximum principal stress value in the stress-strain curve drops or the water pressure value suddenly changes, it indicates that the rock sample has been damaged.

Compared with the prior art, the advantageous effects of the present disclosure are as following:

Compared with traditional indoor uniaxial compression tests and conventional triaxial compression tests, the simulation method provided by the present disclosure can more accurately reflect the stress path during the actual water storage process of the water storage roadway; compared with the true triaxial creep test, it can greatly reduce the number of test specimens and obtain more test data in the same time.

According to the simulation method provided by the present disclosure, by keeping the maximum principal stress σ1, the intermediate principal stress σ2 and the minimum principal stress σ3 unchanged, applying single-sided dynamic water pressure to the rock sample through the front loading head, and applying dynamic load to the rock sample through the impact transmission rod, the stress environment of the actual water storage roadway during the water storage process can be reflected truly, and the triaxial stress state in the actual working condition in the laboratory environment can be known, thus offering a certain reference for the mechanical behavior and failure mode of the rock in the actual working condition.

### BRIEF DESCRIPTION OF THE DRAWINGS

The present invention will be further described with reference to the drawings.

FIG. 1 is a flowchart of a triaxial stress simulation method for water storage roadways in abandoned mine pumped storage according to the present disclosure;

FIG. 2 is a schematic diagram of the force direction of the uniaxial compression for the rock sample of the present disclosure;

FIG. 3 is a force diagram of the triaxial stress of the rock sample according to the present disclosure;

FIG. 4 is a curve chart of uniaxial compression of the rock sample according to the present disclosure;

FIG. 5 is the stress path diagram of the true triaxial seepage disturbance loading for the rock sample in the present disclosure;

FIG. 6 is the stress-time curve of the true triaxial seepage disturbance loading for the rock sample according to the present disclosure;

FIG. 7 is the water pressure-time curve of the true triaxial seepage disturbance loading of the standard rock sample for the rock sample according to the present disclosure;

FIG. 8 is a perspective view of the combined structure of the confining chamber and related parts of the present disclosure;

FIG. 9 is a vertical sectional view of the combined structure shown in FIG. 8;

Reference numbers in the drawings: 1—top cover, 2—clamp segment, 3. clamp, 4—square base plate, 5—stroke cylinder, 6—impact transmission rod, 7—vertical loading rod, 8—horizontal loading rod, 9—longitudinal loading rod, 10—reset spring, 11—circular limiting part, 12—base, 13—cylinder body, 14—positioning column, 15-top loading head, 16—front loading head, 17—rear loading head, 18—water channel, 19-rock sample, 20—square groove.

### DETAILED DESCRIPTION OF THE EMBODIMENTS

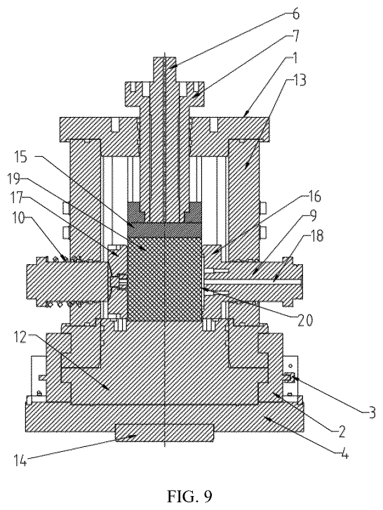

The true triaxial dynamic loading system required by the present disclosure includes a workbench, a main frame, a confining chamber, a vertical loading unit, a horizontal loading unit, a longitudinal loading unit, a lifting device, and a feeding and discharging platform. Combining with FIG. 8 and FIG. 9, the present disclosure mainly introduces the structure of the confining chamber and its related components.

The confining chamber is arranged on the workbench through an adjustable linear transfer mechanism. Specifically, the confining chamber includes a base 12, a cylinder body 13, and a top cover 1. The base 12 is fixed on the upper surface of the linear transfer mechanism, and the base 12 is of a cone frustum structure. The cylinder body 13 is of a cylindrical structure with two open ends and is vertically arranged above the base 12. The upper end of the cylinder body 13 is fixedly sealed with the top cover 1, and the lower end of the cylinder body 13 is sealed with the base 12 and is fixedly connected to the base 12 through a clamping component.

The confining chamber is connected to the water supply device through pipelines. After high-pressure water is filled inside the confining chamber, rock sample 19 can simulate uniaxial loading tests under dynamic and static load confining pressure.

The clamping component includes fours clamp segments 2 and a clamp 3. Each of the clamp segments 2 is in the shape of a 1/4 circular arc with a groove on the inner side. The four clamp segments 2 are spliced into a circular ring and clamped at the connection between the base 12 and the lower end of the cylinder body 13. The clamp 3 is sleeved on the outside of the four clamp segments 2, and the clamping force of the clamp 3 tightens the four clamp segments 2 to fix and connect the lower end of the cylinder body 13 to the base 12.

The vertical loading unit includes an impact loading hydraulic cylinder, top loading hydraulic cylinders, a top loading head 15, and an impact transmission rod 6. The impact loading hydraulic cylinder is vertically and fixedly installed at the center position of the top of the main frame. Four top loading hydraulic cylinder are provided, and the four top loading hydraulic cylinders 52 are vertically and regularly arranged on the periphery of the impact loading hydraulic cylinder. Both the impact loading hydraulic cylinder and the top loading hydraulic cylinders are supplied and returned oil by a hydraulic station.

The top loading head 15 is arranged on the upper part of the inner side of the confining chamber through a vertical loading rod 7. The impact transmission rod 6 is coaxially arranged inside the vertical loading rod 7, and the impact transmission rod 6 cooperates in a sliding mode with the vertical loading rod 7 outside it. The impact load hydraulic cylinder applies an impact load to the top loading head 15 through the impact transmission rod 6, and then the top loading head 15 applies a vertical static load to the rock sample 19. The impact transmission rod 6 applies a vertical impact load to the rock sample 19 through the top loading head 15.

The horizontal loading unit includes loading oil cylinders located on the left side and right side, a left loading head, and a right loading head. The left loading oil cylinder and the right loading oil cylinder are symmetrically and fixedly installed on the left side and right side of the main frame. Both the left loading oil cylinder and the right loading oil cylinder are supplied and returned oil by a hydraulic station.

The left loading head and the right loading head are arranged opposite each other from left to right inside the confining chamber through horizontal loading rods 8. Specifically, the two horizontal loading rods 8 are coaxially passed through the left wall and the right wall of the confining chamber, and each horizontal loading rod 8 cooperates in a sliding mode with the side walls of the confining chamber along its axial direction. The side of the left loading head and the side of the right loading head that away from to each other are fixedly connected to the opposite ends of two horizontal loading rods 8. the ends of the two horizontal loading rods 8 located outside the confining chamber are respectively inserted and cooperated with the two horizontal external loading heads at the ends of the horizontal transmission rod. The left loading oil cylinder and the right loading oil cylinder respectively drive two horizontal external loading heads to move synchronously relative to each other. The horizontal loading rods 8 drive the left loading head and the right loading head to move synchronously relative to each other to load the rock sample 19 laterally.

The vertical loading unit includes an impact transmission rod, and the longitudinal loading unit includes a front loading oil cylinder, a rear loading oil cylinder, a front loading head 16, and a rear loading head 17. The front loading head 16 and the rear loading head 17 are arranged opposite each other from front to rear inside the confining chamber through a longitudinal loading rod 9, the front loading oil cylinder and the rear loading oil cylinder are symmetrically arranged on the front side and rear side of the main frame through a rotating arm, and static loads are synchronously applied to the front loading head and the rear loading head.

A square groove 20 is formed on the end face of the front loading head, and a water channel 18 is arranged inside the longitudinal loading rod connected to the front loading head. The water channel is connected to a water supply device for providing high-pressure water into the square groove.

The positioning column 14 is vertically fixed at the center of the bottom of the square base plate 4, and the lower end of the positioning column 14 can be inserted into the positioning hole, so that the square base plate 4 is fixedly connected to the workbench. Four stroke cylinders 5 are respectively installed on the upper surfaces of the four corners of the square base plate 4. The four stroke cylinders 5 respectively drive four rollers to be stored inside the square base plate 4, the square base plate 4 descends to its bottom and fits the surface of the workbench, the positioning column is located in the positioning hole of the workbench, and the square base plate 4 is fixed on the workbench.

Two horizontal loading rods 8 and two longitudinal loading rods 9 are respectively sleeved by a reset spring 10, all of the reset spring 10 are located on the outer side of the confining chamber. Both the horizontal loading rods 8 and the longitudinal loading rods 9 are provided with annular limiting parts that limit the reset spring 10. In the unloaded state, the reset spring 10 drives the corresponding horizontal loading rods 8 or longitudinal loading rods 9 to move outward, separating the left loading head, right loading head, front loading head, and rear loading head from the rock sample. Before placing the rock sample inside the confining chamber, the left loading head, right loading head, front loading head, and rear loading head are in an expanded state, making it easier to place the rock sample inside the confining chamber The rock sample of the present disclosure adopts cube sample.

Next, the triaxial stress simulation method for water storage roadways in abandoned mine pumped storage is described in detail.

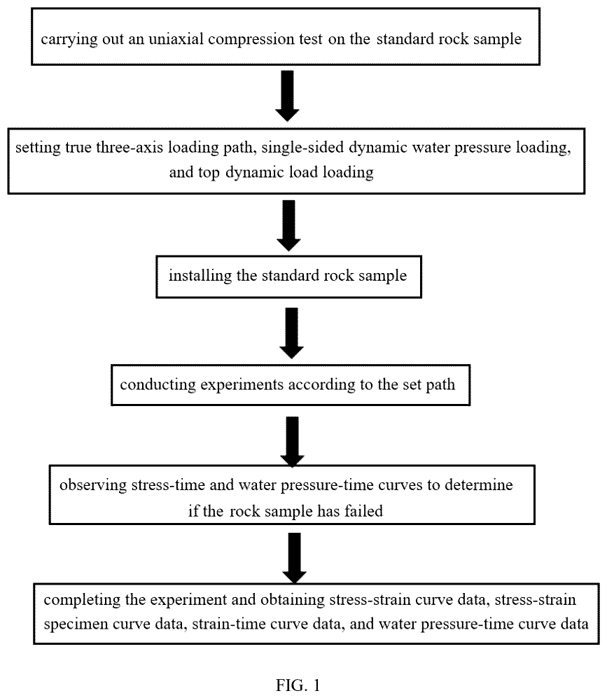

The triaxial stress simulation method suitable for water storage roadways in abandoned mine pumped storage, as shown in FIG. 1, specifically includes the following steps:

Step 1: preparing several rock samples with the same shape, size, and cube shape; obtaining natural rock samples from the engineering site and processing into cubic shapes through drilling, cutting, and grinding, wherein the specifications of the rock samples are 150 mm×150 mm×150 mm, and the length error of the rock samples does not exceed 0.1 mm, the allowable deviation of the unevenness of the two end faces of the rock samples is ±0.02 mm. Selecting rocks with similar wave velocities as test rock samples by a acoustic detection system; when necessary, the loading direction can be identified by marking the rock sample, such as the loading in the X-axis direction, Y-axis direction and Z-axis direction involved in the disclosure.

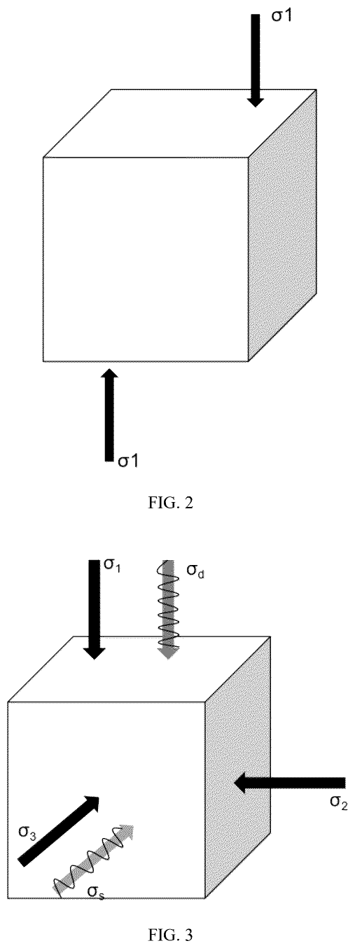

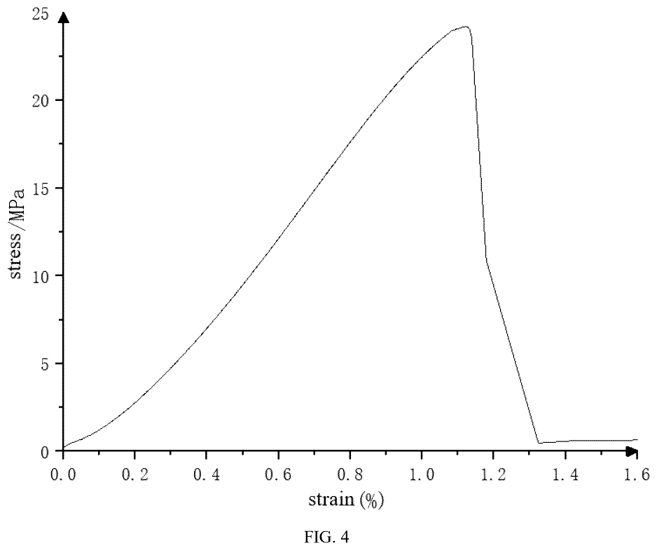

Step 2: selecting one of the rock samples for uniaxial compression testing (as shown in FIG. 2) to obtain its maximum failure load, providing a reference for subsequent loading. The obtained uniaxial compression stress-strain curve is shown in FIG. 4.

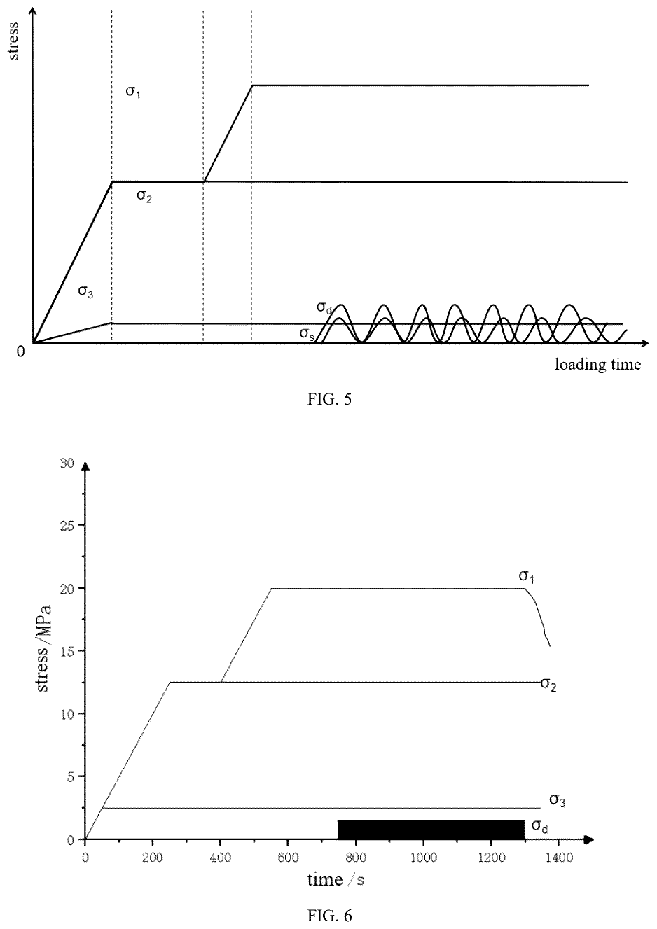

Step 3, selecting another rock sample and installing it in the confining chamber, applying preload to the rock sample for fixation, wherein the loading method is shown in FIG. 3, where σd and σs represent dynamic load and dynamic water pressure, respectively; the loading path is shown in FIG. 5, and the rock sample is a regular cube, ensuring that it can carry out true triaxial test. Fixing the cubic rock sample and applying preload of 10 kN to the rock sample.

As shown in FIG. 3, loading true triaxial stresses σ1, σ2, σ3 independently on cubic rock samples, where σ1 is the maximum principal stress, σ2 is the intermediate principal stress, and σ3 is the minimum principal stress; σ1, σ2, and σ3 are loaded to 50% of the initial principal stress level; adopting the stress loading control method, loading and maintaining true triaxial stresses σ1, σ2, σ3 on the cubic rock sample synchronously and independently by loading method suggested in the technical specification for true triaxial test of rock specimen until the three stresses reach the set initial principal stress level, where.

Step 4: keeping the intermediate principal stress σ2 constant and loading the maximum principal stress σ1 until it reaches the set maximum principal stress level; adopting stress loading control method, loading at the same loading rate in the step 3 to the maximum principal stress σ1 until reaching the set maximum principal stress level, wherein the set maximum principal stress level is 80% of the maximum failure load of the rock sample measured in the step 2.

Step 5: keeping the maximum principal stress σ1, intermediate principal stress σ2, and minimum principal stress σ3 constant, applying a suitable single-sided dynamic water pressure to the rock sample through the front loading head (such as sine wave loading, with a wave trough of 0.1 MPa, a wave peak of 2 MPa, and a frequency of 0.01 Hz) (which can be changed according to experimental needs). Applying water pressure and simultaneously loading impact transmission rod to apply dynamic load on rock sample. (such as sine wave loading, with a wave trough of 0 and a wave trough is 5% of the maximum failure load of the rock sample obtained in the step 2, and the frequency is 5 Hz.) (which can be changed according to experimental needs)

Step 6: observing and recording; recording the stress-strain curve data obtained in the test, and determining whether the stress-strain curve data, strain-time curve data and water pressure-time curve data are needed according to the research situation, wherein the stress-strain curve data can quickly show the true triaxial strength of the test, the strain-time curve data is helpful for the deformation and failure of engineering practice, the stress-strain curve data can reflect the overall mechanical properties of the test rock, and the water pressure-time curve can reflect the changes in internal cracks of the rock.

Ending the test when the rock sample in the true triaxial dynamic loading system has failed, and observing the failure forms of the rock sample. It is preferred to determine whether the rock sample has failed by observing the stress-strain curve and the change of the water pressure-time.

Step 7: combined with theoretical knowledge, obtaining the failure strength and failure form of the surrounding rock of water storage roadways in abandoned mine pumped storage under different depths and water pressures.

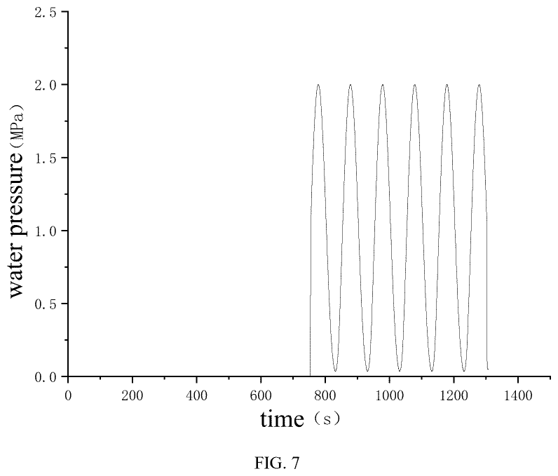

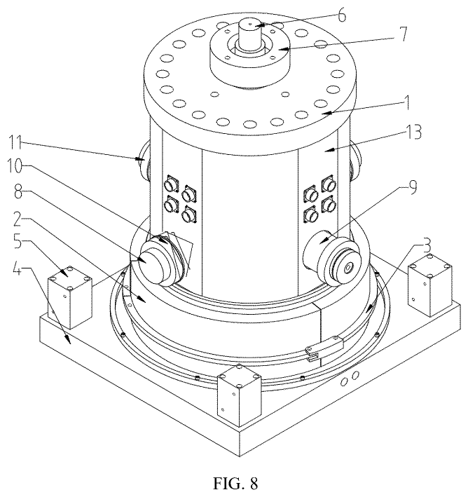

The loading path of this embodiment and the obtained stress-time curve of cyclic loading are shown in FIG. 6, and the water pressure-time curve is shown in FIG. 7.

In summary, the present disclosure can simulate the stress process of surrounding rock during the water storage process of abandoned mine water storage roadways in a more realistic way. Although the present disclosure focuses on abandoned mines, it can also be applied to other similar scenarios (such as underground reservoirs, roadway surrounding rock, etc.) or under different geological conditions, as known to those skilled in the art.

The parts not described in the present invention can be implemented by referring to existing technologies.

Certainly, the above descriptions are merely preferred embodiments of the present disclosure. The present disclosure is not limited to the above embodiments listed. It should be noted that, all equivalent replacements and obvious variations made by any person skilled in the art under the teaching of the specification fall within the essential scope of the specification and shall be protected by the present disclosure.

What is claimed is:

1. A triaxial stress simulation method for water storage roadways in abandoned mine pumped storage, comprising following steps in sequence:
a, preparing a cube sample as a rock sample, and carrying out an uniaxial compression test on the rock sample to obtain a maximum failure load of the rock sample;
b, installing another rock sample in a true triaxial dynamic loading system, wherein the true triaxial dynamic loading system comprises a workbench, a main frame, a confining chamber, a vertical loading unit, a horizontal loading unit, a longitudinal loading unit, a lifting device, and a feeding and discharging platform; the vertical loading unit comprises an impact transmission rod; the longitudinal loading unit comprises a front loading oil cylinder, a rear loading oil cylinder, a front loading head, and a rear loading head; the front loading head and the rear loading head are arranged opposite each other from front to rear inside the confining chamber through a longitudinal loading rod, the front loading oil cylinder and the rear loading oil cylinder are symmetrically arranged on a front side and a rear side of the main frame through a rotating arm, and static loads are synchronously applied to the front loading head and the rear loading head;
a square groove is formed on an end face of the front loading head, and a water channel is arranged inside the longitudinal loading rod connected to the front loading head, the water channel is connected to a water supply device for providing high-pressure water into the square groove;
c. applying a preload to fix the rock sample in the true triaxial dynamic loading system; then applying a maximum principal stress σ1, an intermediate principal stress σ2, and a minimum principal stress σ3 to the rock sample through the vertical loading unit, the horizontal loading unit, and longitudinal loading unit, respectively; during an initial loading process, the maximum principal stress σ1 and the intermediate principal stress σ2 are 50% of the maximum failure load specified in step a;
the minimum principal stress σ3 is 10% of the maximum failure load specified in step a; after the loading stabilizes, the maximum principal stress σ1 is adjusted to 80% of the maximum failure load specified in step a;
d, keeping the maximum principal stress σ1, the intermediate principal stress σ2 and the minimum principal stress σ3 unchanged, applying single-sided dynamic water pressure to the rock sample through the front loading head, and simultaneously applying dynamic load to the rock sample through the impact transmission rod;
e. observing and recording; ending the test when the rock sample in the true triaxial dynamic loading system has failed, recording obtained stress-strain curve data, strain-time curve data, and water pressure-time curve data, and observing a failure mode of the rock sample;
f, combined with theoretical knowledge, obtaining the failure strength and failure form of the surrounding rock of water storage roadways in abandoned mine pumped storage under different depths and water pressures.

2. The triaxial stress simulation method for water storage roadways in abandoned mine pumped storage according to claim 1, wherein in step a, a specification of the rock sample is 150 mm×150 mm×150 mm.

3. The triaxial stress simulation method for water storage roadways in abandoned mine pumped storage according to claim 1, wherein in step b, the vertical loading unit further comprises an impact loading hydraulic cylinder, top loading hydraulic cylinders, and a top loading head; the impact loading hydraulic cylinder is vertically and fixedly installed at a center of a top of the main frame, a plurality of the top loading hydraulic cylinders are provided, all of the top loading hydraulic cylinders are regularly arranged around the impact loading hydraulic cylinder, and execution ends of the top loading hydraulic cylinders are fixedly;
the top loading head is arranged on an upper part of an inner side of the confining chamber through a vertical loading rod, the impact transmission rod is coaxially arranged inside the vertical loading rod, the force collecting block applies static load to the top loading head through the vertical loading rod, and the impact loading hydraulic cylinder applies impact load to the top loading head through the impact transmission rod, wherein the impact load is true triaxial stress.

4. The triaxial stress simulation method for water storage roadways in abandoned mine pumped storage according to claim 1, wherein in step c, preload in Z-axis direction is applied to the rock sample through the vertical loading unit, and the preload in the Z-axis direction is 10 kN; then preload in X-axis direction and preload in Y-axis direction are applied to the rock sample by the longitudinal loading unit and the horizontal loading unit respectively, and the preload in X-axis direction and the preload Y-axis direction is 10 kN, respectively.

5. The triaxial stress simulation method for water storage roadways in abandoned mine pumped storage according to claim 1, wherein in step d, the dynamic load applied to the rock sample by the impact transmission rod is 5% of the maximum failure load in step a.

6. The triaxial stress simulation method for water storage roadways in abandoned mine pumped storage according to claim 1, wherein in step e, whether the rock sample is damaged is determined by stress-strain curve and the water pressure-time curve.

7. The triaxial stress simulation method for water storage roadways in abandoned mine pumped storage according to claim 6, wherein when a maximum principal stress value in the stress-strain curve drops or the water pressure value suddenly changes, indicating that the rock sample has been damaged.
